(19) United States
(12) Patent Application Publication
(10) Pub. No.: US 2004/0186999 A1
(43) Pub. Date: Sep. 23, 2004

(54) Anonymous fingerprinting using bilinear Diffie-Hellman problem

(75) Inventors: Myungsun Kim, Seoul (KR); Kwangjo Kim, Seoul (KR)

Correspondence Address: BACON & THOMAS, PLLC, 625 SLATERS LANE, FOURTH FLOOR, ALEXANDRIA, VA, 22314

(73) Assignee: Information And Communications University Educational Foundation, Seoul (KR)

(21) Appl. No.: 10/726,595

(22) Filed: Dec. 4, 2003

(30) Foreign Application Priority Data
Mar. 19, 2003 (KR) 10-2003-0017022

(51) Int. Cl. A61B 5/103; A61B 5/117; H04L 9/00

(52) U.S. Cl. 713/171

(57) ABSTRACT

A fingerprints embedment system employing an anonymous fingerprinting method using a bilinear Diffie-Hellman problem includes three participants, introduces system parameters and generates a public key and a secret key of each of the first and the second participant. The anonymous fingerprinting method registers information on the first participant to a third participant based on the system parameters and the public and the secret key of the first participant, wherein the third participant issues a certificate based on the information of the first participant and authenticates a fairness of the first participant based on the certificate. Thereafter, the anonymous fingerprinting method embeds fingerprints into a digital content to be bought by the first participant and, when an illegal duplicate of the digital content or an illegally redistributed duplicate is found, identifies a traitor, which illegally duplicates the digital content or redistributes the illegally duplicated digital content, with the first participant.

### FIELD OF THE INVENTION

The present invention relates to a fingerprinting scheme; and, more particularly, to an anonymous fingerprinting scheme capable of preserving anonymity of a buyer and reducing a key size of algorithm without a collusion attack problem.

### BACKGROUND OF THE INVENTION

In the progress of computer networks and the development of Internet, protection of digitally stored information property has become a crucial problem that should be solved. Many schemes such as a fingerprinting scheme and a watermarking scheme have been proposed for technically supporting the copyright protection of digital data.

The watermarking scheme serves as one of the reasonable alternatives for solving several problems such as a piracy, an illegal duplicate and an illegal distribution thereof in a manner that an owner of a digital content embeds specific information in the digital content and extracts the specific information therefrom.

On the other hand, the fingerprinting scheme is one of cryptography for protecting copyright of a digital content, in which information on a buyer is embedded by the watermarking scheme. In a conventional fingerprinting scheme, a buyer embeds information on the buyer in a digital content and a merchant examines an illegal duplicate or an illegally redistributed duplicate to trace an illegal buyer or re-distributor based on the buyer's information embedded in the illegally redistributed duplicate. Further, in the fingerprinting scheme, the merchant can identify an original buyer of the illegally redistributed duplicate, referred to as a traitor, thereby deterring a buyer from illegally redistributing the digital content.

The conventional fingerprinting scheme is usually divided into two classes, i.e., a symmetric fingerprinting and an asymmetric fingerprinting.

In the symmetric fingerprinting, fingerprints are embedded in a digital content only by a merchant. However, even if the merchant recognizes an identity of a traitor from the digital content, the merchant cannot convince a third party, especially, a registration authority, who the traitor is.

In the asymmetric fingerprinting, fingerprints are embedded in a digital content by an interactive protocol between a buyer and a merchant. When a transaction between the buyer and the merchant is completed, only the buyer has a fingerprinted copy. If the merchant has found an illegally distributed duplicate somewhere, the merchant can distinguish a traitor from other buyers and prove a third party, especially, a registration authority, who the traitor is.

However, for two aforementioned fingerprinting schemes, there is a possibility of infringing privacy of a buyer because a merchant requires information about the buyer. Further, if a buyer purchases digital items, especially, through an open network, information on the buyer, e.g., a shopping behavior and a personal profile, may be revealed to the public, which in turn can be commercially abused through networks.

Thus, when a buyer purchases a fingerprinted digital content, it is desirable that the buyer remains anonymous as long as the buyer does not illegally distribute the digital content. For this purpose, an anonymous asymmetric fingerprinting (in short, an anonymous fingerprinting) has been proposed.

The anonymous fingerprinting retains the asymmetric property as a sort of the asymmetric fingerprinting. In the anonymous fingerprinting, a buyer can purchase a fingerprinted digital content without revealing a profile of the buyer to a merchant, while a merchant can detect a traitor when finding an illegally redistributed duplicate.

However, the conventional fingerprinting scheme, especially, the anonymous fingerprinting, does not take account of computational capability of a buyer. In other words, it is not easy to practically use algorithm for supporting the conventional fingerprinting scheme because a key size for the algorithm is too large.

Further, in the fingerprinting scheme, there is a probability for collusion attack. That is, a collusion group can obtain a multiplicity of digital contents having different fingerprints and then compare each other to capture positions where original fingerprints are embedded. Then the collusion group can remove the original fingerprints and interpolate gaps to thereby resell the digital contents without worrying about being traced.

### SUMMARY OF THE INVENTION

It is, therefore, an object of the present invention to provide an anonymous fingerprinting method and apparatus using bilinear pairings, which is capable of preserving anonymity of a buyer as long as the buyer does not illegally distribute digital contents and reducing a key size of algorithm without risking a collusion attack problem.

In accordance with an aspect of the present invention, there is provided an anonymous fingerprinting method using a bilinear Diffie-Hellman problem, in a fingerprints embedment system that includes three participants, including the steps of: (a) introducing system parameters shared by a first and a second participant, storing the system parameters in a memory of each of the first and the second participant and generating a public key and a secret key of the first participant; (b) registering information on the first participant to a third participant based on the system parameters and the public and the secret key of the first participant, wherein the third participant issues a certificate based on the information on the first participant; (c) at the second participant, authenticating a fairness of the first participant based on the certificate; (d) embedding fingerprints into a digital content to be bought by the first participant; and (e) when an illegal duplicate of the digital content or an illegally redistributed duplicate is found, identifying a traitor, who illegally duplicates the digital content or redistributes the illegally duplicated digital content, with the first participant based on the fingerprints embedded in the digital content.

In accordance with another aspect of the present invention, there is provided an anonymous fingerprinting apparatus using a bilinear Diffie-Hellman problem, including: a registration authority; a buyer; and a merchant, wherein the apparatus performs the steps of: introducing system parameters shared by a first and a second participant, storing the system parameters in a memory of each of the first and the second participant and generating a public key and a secret key of each of the first and the second participant; registering information on the first participant to a third participant based on the system parameters and the public and the secret key of the first participant, wherein the third participant issues a certificate based on the information of the first participant; at the second participant, authenticating a fairness of the first participant based on the certificate; embedding fingerprints into a digital content to be bought by the first participant; and when an illegal duplicate of the digital content or an illegally redistributed duplicate is found, identifying a traitor, who illegally duplicates the digital content or redistributes the illegally duplicated digital content, with the first participant.

### BRIEF DESCRIPTION OF THE DRAWINGS

The above and other objects and features of the present invention will become apparent from the following description of preferred embodiments given in conjunction with the accompanying drawings, in which:

FIG. 1 shows a flow chart for illustrating an anonymous fingerprinting scheme using a B-DH (bilinear Diffie-Hellman) problem in accordance with the present invention;

FIG. 2 represents a schematic block diagram for representing the anonymous fingerprinting scheme using B-DH problem;

FIG. 3 depicts a flow chart for illustrating a setting process of the anonymous fingerprinting scheme using B-DH problem;

FIG. 4 provides a flow chart for illustrating a registering process of the anonymous fingerprinting scheme using B-DH problem;

FIG. 5 offers a flow chart for illustrating a buyer authenticating proces of the anonymous fingerprinting scheme using B-DH problem;

FIG. 6 is a flow chart for illustrating a fingerprints embedding process of the anonymous fingerprinting scheme using B-DH problem; and

FIG. 7 describes a flow chart for illustrating an identifying process of the anonymous fingerprinting scheme using B-DH problem.

### DETAILED DESCRIPTION OF THE PREFERRED EMBODIMENTS

An anonymous fingerprinting scheme in accordance with a preferred embodiment of the present invention makes use of a bilinear map on an elliptic curve to construct a group G, in which a C-DH (computational Diffie-Hellman) problem is intractable but a D-DH (decisional Diffie-Hellman) problem is tractable. However, since in the D-DH problem, it is too easy to build cryptosystems, the security of the anonymous fingerprinting scheme in accordance with the present invention is intrinsically based on an intractability of the C-DH problem called a B-DH (bilinear Diffie-Hellman) problem.

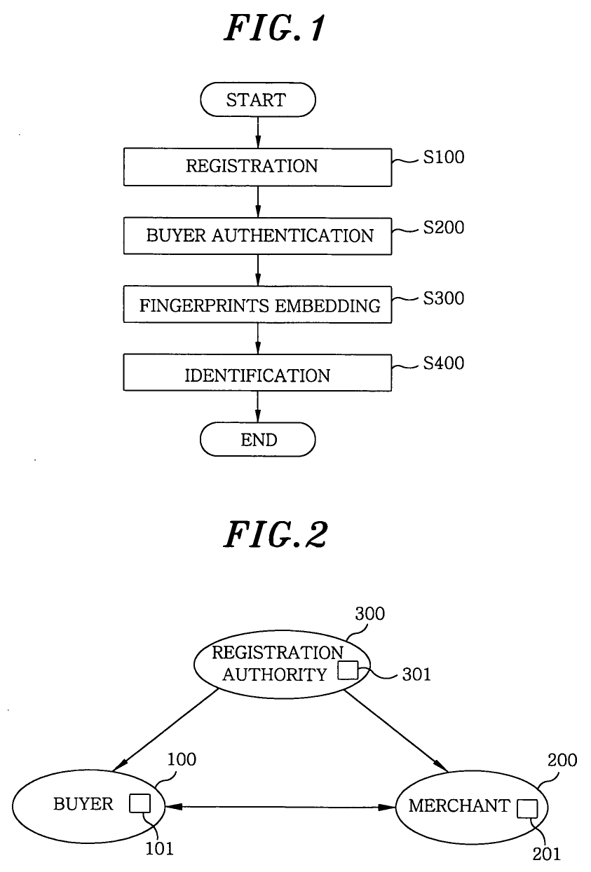

The anonymous fingerprinting scheme using B-DH problem in accordance with the present invention includes following three procedures: a registration procedure (step S100), a fingerprinting procedure (steps S200 and S300) and an identification procedure (step S400), wherein the registration procedure involves a key generation process and the fingerprinting procedure is divided into a buyer authentication process (step S200) and a fingerprints embedding process (step S300), as shown in FIG. 1.

FIG. 2 represents an anonymous fingerprinting scheme in accordance with the present invention, which includes a buyer 100, a merchant 200 and a registration authority 300, each of which has a storage medium, e.g., a memory, and an operating unit, e.g., a CPU, as participants of the anonymous fingerprinting scheme. Each of the participants, which may be a computer system or an actual user, communicates remotely through any kind of communications network or other techniques. Information to be transferred between each of the participants may be stored or detained in various types of storage media.

The registration authority 300, which has an operating unit (not shown) for computing algorithms and a storage medium 301 for storing computed results or specific information, introduces system parameters to be shared and utilized by the buyer 100 and the merchant 200. Also, the registration authority 300 produces a public key and a secret key of the buyer 100 based on the system parameters and then provides corresponding keys to the buyer 100 through secure channels.

Further, the registration authority 300 issues a certificate so that the buyer 100 can prove fairness for itself to the merchant 200. Moreover, when the merchant 200 detects a clue for a buyer to be a traitor and presents the registration authority 300 with a proof that the buyer is the traitor, the registration authority 300 ascertains whether the buyer is truly the traitor.

The buyer 100, which has an operating unit (not shown) for computing algorithms and a storage medium 101 for storing computed results or specific information, registers information of the buyer such as personal profile to the registration authority 300 by using the public and the secret key of the buyer 100 provided from the registration authority 300. The information on the buyer 100 becomes grounds that the merchant 200 can distinguish the buyer 100 from another buyers when the merchant 200 finds an illegal digital content. That is, when the illegal digital content is found, the information on the buyer 100 can be used for the merchant 200 to ascertain who an original buyer of the illegal digital content is.

The buyer 100 authenticates fairness for itself to the merchant 200 by using the secret and the public key of the buyer 100 and the certificate provided from the registration authority 300. Further, the buyer 100 participates in the fingerprints embedding process through an interactive protocol with the merchant 200, while concealing an identity of the buyer 100, after being determined to be fair in the buyer authentication process.

The merchant 200, which has an operating unit (not shown) for computing algorithms and a storage medium 201 for storing computed results or specific information, examines information presented by the buyer 100 to verify fairness of the buyer 100. Further, the merchant 200 participates with the buyer 100 in the fingerprints embedding process.

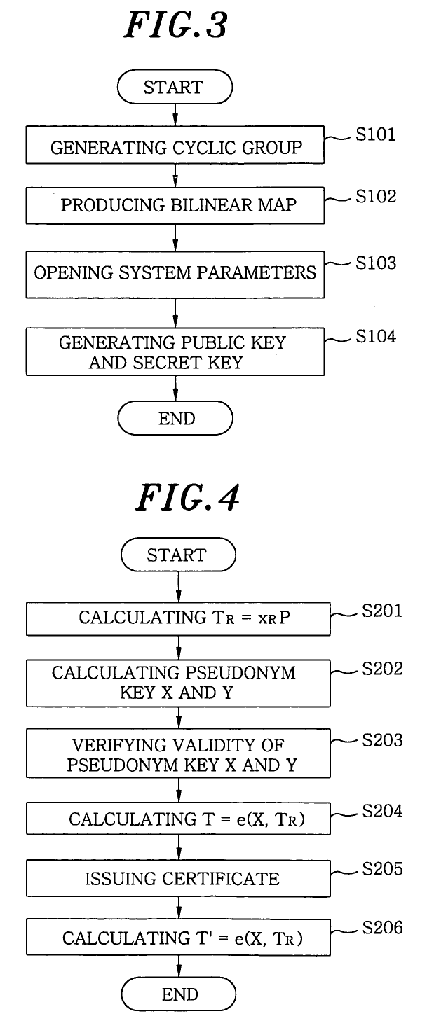

Hereinafter, the anonymous fingerprinting scheme using B-DH problem of the present invention will be described with reference to FIGS. 3 to 6, in detail.

FIG. 3 depicts a flow chart for illustrating the key generation process of the registration procedure in the anonymous fingerprinting scheme using B-DH problem.

At step S101, the registration authority 300 generates cyclic groups G₁ and G₂, each of which is of a prime order m, and then takes an arbitrary generator P out of the cyclic group G₁, wherein the cyclic group G₁ is an elliptic curve group and the cyclic group G₂ is a cyclic multiplicative group.

At step S102, the registration authority 300 produces a bilinear map e on the two cyclic groups G₁ and G₂ as follows:

e: G₁×G₁→G₂ Eq. 1.

At step S103, the registration authority 300 stores system parameters such as G₁, G₂ and P in the storage medium 301 and opens the system parameters so that the buyer 100 and the merchant 200 can share and use them.

At step S104, the registration authority 300 selects random values s₁, s₂ and s₃ corresponding to G₂ to generate a secret key {s₁, s₂, s₃} of the buyer 100 and then calculates a public key y_{B} of the buyer 100 as follows:

y_{V}=e(P, p)^{s}^{₁}^{s}^{₂}^{s}^{₃} Eq. 2

and then the secret key {s₁, s₂, s₃} and the public key y_{B} are forwarded to the buyer 100 to be stored in the storage medium 101 of the buyer 100.

Hereinafter, the registration procedure after the key generation process in the anonymous fingerprinting scheme using B-DH problem will be described as shown in FIG. 4.

At step S201, the registration authority 300 selects an arbitrary random value. xᵣ corresponding to the cyclic group G₂ based on the system parameters, calculates a confidential value T_{R} as follows:

T_{R}=X_{R}P Eq. 3

, and then sends the confidential value T_{R} to the buyer 100, wherein T_{R} is used to make it sure that x_{R} is safely sent to the buyer 100.

At step S202, the buyer 100 computes pseudonym keys X and Y by using the secret key {s₁, s₂, s₃} as follows:

X=s₁s₂P Eq. 4, and

Y=s₁s₂s₃P+T_{R} Eq. 5

and then sends the pseudonym keys X and Y to the registration authority 300.

At step S203, on receiving the pseudonym keys X and Y, the registration authority 300 verifies validity of the pseudonym keys X and Y as follows:

e(Y, P)=y_{B}·e(P, T_{R}) Eq. 6.

At step S204, if the pseudonym keys X and Y determined to be valid, the registration authority 300 calculates T as follows:

T=e(X, T_{R}) Eq. 7

and stores calculated result in the storage medium 301, wherein T is an intermediate value for judging whether or not the buyer 100 is an owner of a secret key corresponding to the pseudonym keys X and Y.

At step S205, the registration authority 300 issues certificates Cert(T) and Cert(Y|x_{R}), which certify respectively validities of T and Y, for proving a fairness of the buyer 100 and then forwards the certificates Cert(T) and Cert(Y|x_{R}) to the buyer 100.

At step S206, on receiving the certificates Cert(T) and Cert(Y|a_{R}), the buyer 100 calculates T′ as follows:

T′=e(X, T_{R}) Eq. 8

wherein T′ is a value for notifying that the buyer 100 is an owner of a secret key corresponding to the pseudonym keys X and Y. Then the buyer 100 views (Y, T′) as a pseudonym pair and safely stores the pseudonym pair (Y, T′) in the storage medium 101.

Next, the buyer authentication process in the anonymous fingerprinting scheme using a B-DH problem will be carried out as shown in FIG. 5.

At step S301, the buyer 100 sends Y, [T′, Cert(T)] and text to the merchant 200, wherein the text represents normal information about a digital content to be fingerprinted.

At step S302, the buyer 100 selects an arbitrary random value k corresponding to G₂, thereby generating a B-DH signature Sig to be embedded as follows:

Sig=Sign(text, s₁, s₂, s₃, x_{R}, k) Eq. 9.

At step S303, the merchant 200 checks fairness of the buyer 100 based on the certificate Cert(T) sent from the buyer 100. If the buyer 100 is determined to be fair, the merchant 200 stores [T′, Cert(T)] as a purchase record of the buyer 100 in the storage medium 201.

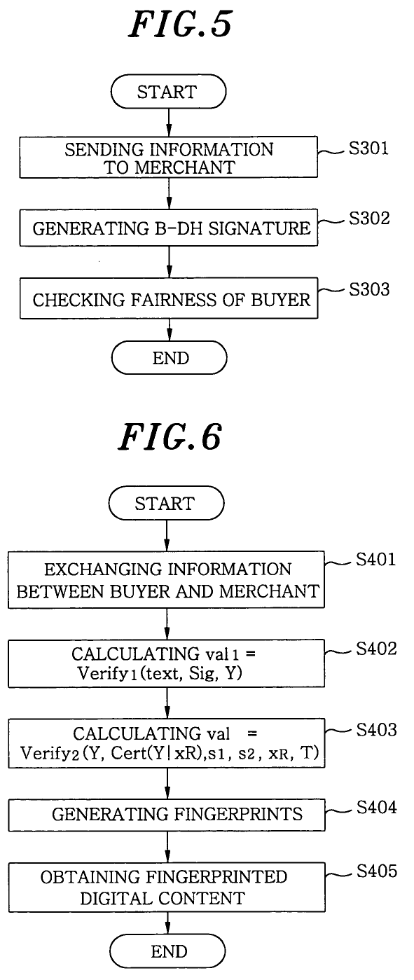

FIG. 6 shows a fingerprints embedding process for the buyer 100 and the merchant 200, which is realized through a secure two-party computation between them.

At step S401, the buyer 100 and the merchant 200 exchange certain information therebetween. That is, the buyer 100 sends x_{R}, Sig, s₁, x₂, Cert(Y|x_{R}) to the merchant 200 and the merchant 200 presents T′, Y, the text and em to the buyer 100, wherein em denotes the digital content to be fingerprinted.

At step S402, a specific value val₁ is generated as follows:

valₙ=Verify₁(text, Sig, Y) Eq. 10

, val₁ being a Boolean variable to be seen by only the merchant 200 when verification of the B-DH signature Sig for the text is completed successfully.

At step S403, a particular value val₂ is generated as follows:

val₂=Verify₂(Y, Cert(Y|x_{R}), s₁, s₂, x_{R}, T′) Eq. 11

val₂ being also a Boolean variable to be seen by only the merchant 200 when the certificate Cert(Y|x_{R}) and the B-DH signature Sig for the text are respectively verified as to be described later.

At step S404, the merchant 200 generates emb as follows:

emb=text|Sig|Y|Cert(Y|x_{R})|s₁|s₂|x_{R}|T′ Eq. 12

to be stored in the storage medium 201, wherein emb represents fingerprints to be embedded into the digital content em.

At step S405, the merchant 200 obtains a fingerprinted digital content em* as follows:

em*=Fing(em, emb) Eq. 13

and then sends the fingerprinted digital content em* to the buyer 100.

As a consequence, the fingerprinted digital content em* is obtained as an output of the two-party computation and is seen by only the buyer 100. However, the buyer 100 cannot get the fingerprinted digital content em* unless both val₁ and val₂ from Eqs. 10 and 11 are true.

Finally, an identification procedure is carried out in case where the merchant 200 detects a clue for a buyer to be a traitor.

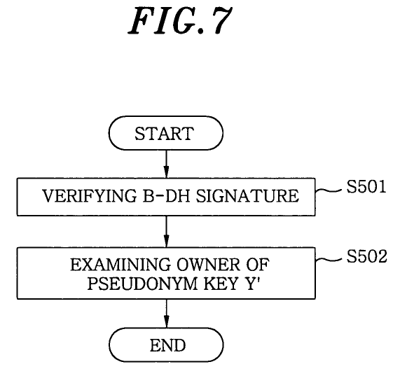

Referring to FIG. 7, at step S501, the merchant 200 verifies the B-DH signature Sig for the text as follows:

T″=e(s₁s₂P, x_{R}P) Eq. 14

and then stores the verified result from Eq. 14 in the storage medium 201, wherein T″ is a value for checking whether or not the buyer 100 is an owner of a secret key corresponding to the pseudonym key Y′.

At step S502, the merchant 200 examines who is an owner of a pseudonym key Y′, as follows:

$$\begin{matrix}e ( {Y}^{′} , P ) = e ( {s}_{1} {s}_{2} {s}_{3} P + {T}_{R} P ) \\ = e ( {s}_{1} {s}_{2} {s}_{3} P + {x}_{R} P , P ) \\ = e ( {s}_{1} {s}_{2} {s}_{3} P , P ) \cdot e ( {x}_{R} P , P ) \\ = {y}_{B} \cdot {e ( P , P )}^{{x}_{R}}\end{matrix} , \qquad Eq . 15$$

, wherein if the pseudonym key Y′ satisfies Eq. 15, the merchant 200 can prove that the owner of the pseudonym key Y′ is the traitor.

That is, only x_{R} is associated with T and Y as seen from Eq. 5 and Eq. 7 and only the registration authority 300 can provide x_{R} such that the result T″ of Eq. 14 is same as the result T of Eq. 7. Further, the buyer 100 cannot produce T′ identical to T without knowing x_{R} in polynomial time and the merchant 200 cannot forge x_{R} because T and Y are certified by only the registration authority 300. Therefore, x_{R} can be used for proving that an owner of the pseudonym key Y′ is same as that of T′. In other words, the merchant 200 can prove that the buyer 100 is a traitor because T″ calculated by the merchant 200 based on T′ provided from the buyer 100 does not correspond to T calculated by the registration authority 300.

While the invention has been shown and described with respect to the preferred embodiments, it will be understood by those skilled in the art that various changes and modifications may be made without departing from the spirit and scope of the invention as defined in the following claims.

What is claimed is:

1. An anonymous fingerprinting method using a bilinear Diffie-Hellman problem, in a fingerprints embedment system that includes three participants, comprising the steps of:
(a) introducing system parameters shared by a first and a second participant, storing the system parameters in a memory of each of the first and the second participant and generating a public key and a secret key of the first participant;
(b) registering information on the first participant to a third participant based on the system parameters and the public and the secret key of the first participant, wherein the third participant issues a certificate based on the information on the first participant;
(c) at the second participant, authenticating a fairness of the first participant based on the certificate;
(d) embedding fingerprints into a digital content to be bought by the first participant; and
(e) when an illegal duplicate of the digital content or an illegally redistributed duplicate is found, identifying a traitor, who illegally duplicates the digital content or redistributes the illegally duplicated digital content, with the first participant based on the fingerprints embedded in the digital content.

2. The method of claim 1, wherein the step (a) includes the steps of:
(a1) generating G₁ and G₂, wherein G₁ is an elliptic curve group and G₂ is a cyclic multiplicative group;
(a2) taking a generator P out of the cyclic multiplicative group G₂;
(a3) calculating a bilinear map e on the groups G1 and G2 as follows:
e: G₁×G₁→G₂
(a4) storing the system parameters in a storage medium of the third participant and opening the system parameters so that the first and the second participant can use them, wherein the system parameters has G₁, G₂ and P;
(a5) selecting a secret key of the first participant out of G₂, wherein the secret key of the first participant is formed of s₁, s₂ and s₃; and
(a6) calculating a public key y_{B} of the first participant as follows:
y_{B}=e(P, P)^{s}^{₁}^{s}^{₂}^{s}^{₃}.

3. The method of claim 2, wherein the step (b) includes the steps of:
(b1) generating a random value x_{R} corresponding to G2;
(b2) calculating a confidential value T_{R} as follows:
T_{R}=x_{R}P
and sending the confidential value T_{R} to the first participant;
(b3) calculating pseudonym keys X and Y of the first participant as follows:
X=s₁s₂P
Y=s₁s₂s₃P+T_{R};
(b4) verifying validity of X and Y as follows:
e(Y, P)=y_{B}·e(P, T_{R});
(b5) calculating T as follows:
T=e(X, T_{R})
and storing T in a memory of the third participant, wherein T is an intermediate value for judging whether or not the first participant is an owner of a secret key corresponding to the pseudonym keys X and Y;
(b6) issuing the certificate, which proves a fairness of the first participant to the second participant, and delivering the certificate to the first participant; and
(b7) at the first participant, calculating T′ as follows:
T′=e(X, T_{R})
and viewing (Y, T′) as a pseudonym pair to safely store the pseudonym pair in a memory of the first participant, wherein T′ is a value for notifying that the first participant is an owner of the secret key corresponding to X and Y.

4. The method of claim 3, wherein the step (c) includes the steps of:
(c1) sending the pseudonym pair and text to the second participant, wherein the text represents normal information about a digital content to be fingerprinted;
(c2) selecting a random value k out of G₂ to generate a B-DH signature Sig for the text, as follows:
Sig=sign(text, s₁, s₂, s₃, x_{R}, k); and
(c3) verifying validity of the certificate and storing T′ and the certificate as a purchase record of the first participant in a memory of the second participant.

5. The method of claim 4, wherein the step (d) includes the steps of:
(d1) at the first participant, sending x_{R}, Sig, s₁, s₂ and the certificate to the second participant and, at the second participant, presenting T′, Y, em and the text to the first participant, wherein em denotes the digital content to be fingerprinted;
(d2) generating a specific value val₁ as follows:
val₁=Verify₁(text, sig, Y)
wherein val₁ is a Boolean variable to be seen by the second participant when verification of Sig is completed;
(d3) generating a particular value val₂ as follows:
val₂=Verify₂(Y, Cert(Y|x_{R}), s₁, s₂, x_{R}, T)
wherein val₂ is a Boolean variable to be seen by the second participant when the certificate and Sig are respectively verified;
(d4) generating emb as follows:
emb=text|Sig|Y|Cert(Y|x_{R})|s₁|s₂|x_{R}|T′
and storing emb in a memory of the second participant, wherein emb represents fingerprints to be embedded into the digital content em; and
(d5) obtaining a fingerprinted digital content em* as follows:
em*=Fing(em, emb).

6. The method of claim 5, wherein the step (e) includes the steps of:
(e1) verifying validity of Sig for the text as follows:
T″=e(s₁s₂P, x_{R}P)
wherein T″ is a value for checking whether the first participant is an owner of a secret key corresponding to the pseudonym key Y′; and
(e2) determining an owner of the pseudonym key Y′ to be the traitor if the pseudonym key Y′ is given as follows:
e(Y′, P)=y_{B}·e(P, P)x_{R}.

7. An anonymous fingerprinting apparatus using a bilinear Diffie-Hellman problem, comprising:
a registration authority;
a buyer; and
a merchant,
wherein the apparatus performs the steps of:
introducing system parameters shared by a first and a second participant, storing the system parameters in a memory of each of the first and the second participant and generating a public key and a secret key of each of the first and the second participant;
registering information on the first participant to a third participant based on the system parameters and the public and the secret key of the first participant, wherein the third participant issues a certificate based on the information of the first participant;
at the second participant, authenticating a fairness of the first participant based on the certificate;
embedding fingerprints into a digital content to be bought by the first participant; and
when an illegal duplicate of the digital content or an illegally redistributed duplicate is found, identifying a traitor, who illegally duplicates the digital content or redistributes the illegally duplicated digital content, with the first participant.
